(19) United States
(12) Patent Application Publication
(10) Pub. No.: US 2010/0317805 A1
(43) Pub. Date: Dec. 16, 2010

(54) POLYMERISABLE NAPHTHOPYRANE DERIVATIVES AND POLYMER MATERIALS OBTAINED FROM THESE DERIVATIVES

(75) Inventors: Sébastien GIBANEL, CHALON SUR SAONE (FR); Jean-Luc POZZO, GUJAN-MESTRAS (FR); Anne PAGNOUX, LE BARP (FR); Marc DOLATKHANI, CESTAS (FR)

Correspondence Address: LUCAS & MERCANTI, LLP, 475 PARK AVENUE SOUTH, 15TH FLOOR, NEW YORK, NY, 10016, US

(73) Assignee: POLYMEREXPERT, Pessac (FR)

(21) Appl. No.: 12/868,500

(22) Filed: Aug. 25, 2010

Related U.S. Application Data
(63) Continuation of application No. 11/569,440, filed on Jan. 11, 2007, now abandoned.

(30) Foreign Application Priority Data
May 21, 2004 (FR) FR0405515

(51) Int. Cl. C08F 34/02; C08F 265/04; C07D 311/78

(52) U.S. Cl. 525/284; 526/268; 549/388

(57) ABSTRACT

The invention relates to novel compounds of formula (I):

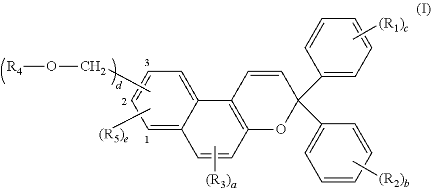

in which
R₁, R₂ and R₃, identical or different, independently represent:
   hydrogen
   a halogen
   a C1 to C15 hydroxy or hydroxyalkyl group
   a C1 to C15 linear or branched alkyl group a, b and c independently lying between 0 and 5.
R₄ is linked to the naphthalene unit at position 1, 2 or 3 via a —CH₂—O— bond and represents:
   either a divalent group which is (co)polymerisable with a monomer; in which case d lies between 1 and 3
   or a monovalent group which is (co)polymerisable with a monomer; in which case d is equal to 2 or 3 and
R₅ is linked to the naphthalene unit at position 1, 2 or 3 and represents:
   hydrogen,
   a halogen,
   a C1 to C15 hydroxyalkyl group,
   a C1 to C15 linear or branched alkyl group,
      and e is an integer lying between 0 and 2 and such that d+e=3.

It also relates to the method for synthesising these compounds and to their use for the manufacture of photochromic or photosensitive polymers.

This is a continuation of U.S. patent application Ser. No. 11/569,440 filed Jan. 11, 2007, which in turn was a National Phase (371) of PCT/FR2005/001266 filed May 20, 2005, which claimed the priority of French Patent Application no. 0405515 filed May 21, 2004. Each of the above identified applications is incorporated herein, by reference.

The invention relates to novel polymerisable naphthopyrane derivatives as well as to polymer materials obtained from these derivatives.

Photochromic materials are materials well known for changing colour recursively when they are exposed to light, for example ultraviolet rays. Numerous applications in the field of ophthalmic optics have been developed around these molecules: darkening lenses of sunglasses, changing the colour of contact lenses, etc. making it possible to protect the retina against damage by ultraviolet rays.

The most widely used photochromic chemical agents are:
spiro-indolino-pyranes: the coloration developed is very intense under little radiation for compounds substituted by one or more of the groups nitro, cyano, amino, alcoxy. The frequent changes between stable shape and excited state, however, rapidly cause degradation of the molecule.
spirobenzothiazolo-benzopyranes and/or spiroindolino-benzothiopyranes: The coloration developed under irradiation lies in the blue range, but the thermal decolouration rate is slow compared with naphthopyranes and the low coloration efficiency remains a major drawback for the use of these substances as a photochromic pigment.
spiro-indolino-oxazines: these compounds have been widely used in materials with variable optical transmission, and have shown remarkable overall properties: coloration, fatigue strength, thermal decolouration rate which are compatible with the desired applications. It should, however, be noted that these properties are restricted to molecules which develop a coloration ranging from blue to green. The chemical modifications making it possible to achieve the colour red lead to a very strong increase in photofatigue.
naphthopyranes: Among naphthopyranes, compounds of the 3,3-diaryl-3H-naphtho[2,1-b]pyrane type present an intense coloration in the yellow-orange-red range, excellent fatigue strength in the presence of phenyl substituents at position 3 and thermal decolouration kinetics in the absence of light irradiation compatible with the applications desired by the inventors of the present invention. Other colours can be obtained with other substituents, at certain temperatures (U.S. Pat. No. 3,567,605, 1971). This type of molecule is a good compromise from the point of view of its photochromic performance: rapid coloration and decolouration in a wide working temperature range (0 to 40° C.), intense coloration in the excited form (U.S. Pat. No. 5,631,720, JP8176139, JP8157467, U.S. Pat. No. 6,113,814, WO97/05213). New derivative molecules such as tetraphenyl naphthodipyranes (U.S. Pat. No. 5,464,567) or indeno-fused naphthopyranes (WO9614596) has been developed in order to extend the emitted spectrum from orange to blue/grey.

Naphthopyranes are a good compromise between intensity of the colour and decolouration rate. Furthermore, their structural change in the excited form is not perturbed by the surrounding polymer matrix and they present good fatigue strength.

The principle of coloration under the action of UV will be summarised below: the ring carrying the oxygen atom opens and the conjugation of the double bonds resulting therefrom leads to the development of colorations.

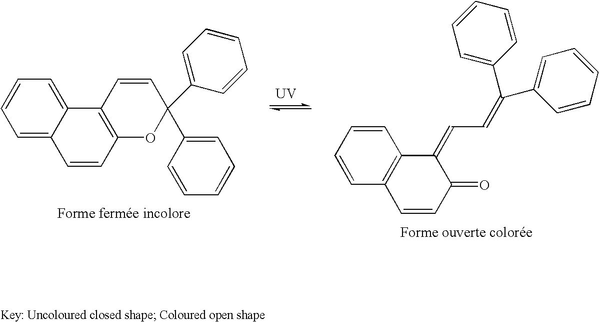

Photochromic materials are generally manufactured from polymer matrices within which the photochromic molecules are dispersed (WO0160811). Since 1990, certain authors have functionalised these molecules in order to render them polymerisable:
Toray Industries developed a polymerisable spirooxazine in 1990 (U.S. Pat. No. 5,166,345), Nat Science Council developed another in 1997 (U.S. Pat. No. 582,187)
Otsuka Kagaku addressed the performance of polymerisable spiropyranes in 1992-1993 (U.S. Pat. No. 5,236,958, U.S. Pat. No. 5,252,742), Mr Yun Ki likewise (US2003099910)
Sola International Holdings has intercalated a spacer between the photochrome and the polymerisable function in order to render it more compatible with the matrix (WO9705213)
Transitions Optical describes in WO03056390 and U.S. Pat. No. 6,113,814 all the techniques known to date for rendering a photochrome polymerisable, but it describes only molecules of the naphthopyrane family carrying a dimethoxyphenyl substituent on carbon No 3 or a polymerisable group linked to the naphthalene via at least one O—CH₂—CH(H or CH₃)—O unit.

The principle of functionalising photochromic molecules in order to render them copolymerisable with the monomers used to synthesise the polymer matrix is not new. In this way, the photochromic molecules are chemically linked to the polymer matrix and they can no longer migrate over time, as a function of the temperature to which the photochromic material is exposed.

The inventors of the present invention have now developed novel derivative molecules of 3,3-diaryl-3H-naphtho[2,1-b]pyranes (naphthopyranes) which permit applications in which these photochromic molecules are in contact with the human body, particularly in dermatology, cosmetics and opthalmological, and whose toxicity is very low or even non-existent owing to the fact that these molecules cannot be assimilated by cells.

Moreover, these molecules could be prepared in a much simpler way compared with the syntheses known in the prior art, particularly by avoiding intermediate purification steps which are particularly intricate and laborious, which makes these novel products industrially viable by avoiding in particular the steps of purifying the synthesis intermediaries obtained, for example as is the case according to the publication Pozzo et al., Langmuir (2002), 18(19), 7096-7101.

The novel polymerisable photochromic molecules of the present invention thus present good coloration in the yellow-orange range, a rapid decolouration rate and a high fatigue strength, and furthermore can be easily prepared industrially.

More precisely, the photochrome of the invention is an at least divalent monomer (co)polymerisable by chain or stepwise polymerisation. It is a substituted 3,3-diaryl-3H-naphtho[2,1-b]pyrane which, as indicated above and as will emerge from the description and the examples, has the advantage that it can be synthesised easily on an industrial scale.

Because of its lower production cost and the relatively good synthesis yield, applications may be envisaged for this molecule in optics, opthalmological, cosmetics, or even in sectors such as textiles, building, etc. Its stability will furthermore be increased commensurately in the corresponding materials, since it is copolymerisable with the products involved in their composition.

These photochromic molecules can thus be copolymerised with all the existing monomers, individually or as a mixture with other polymerisable photochromes described in the literature.

The invention therefore relates, according to a first aspect, to a novel family of polymerisable naphthopyrane derivatives.

According to a second aspect, the invention relates to the process for synthesising these novel compounds.

According to a third aspect, the invention relates to novel products resulting from the polymerisation or copolymerisation of the naphthopyranes of the invention as well as to products obtained by chemical modification of a monomer or an oligomer by reaction with a naphthopyrane of the invention. The invention finds applications in various fields in which ultraviolet filtration and/or photochromic properties are desired.

More precisely, according to an essential characteristic of its first aspect, the invention relates to a compound of the polymerisable substituted 3,3-diaryl-3H-naphtho[2,1-b]pyrane type, characterised in that is corresponds to formula (I):

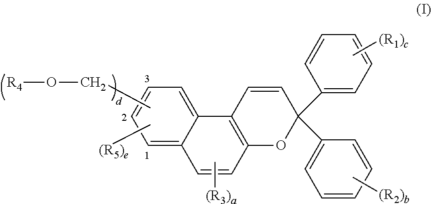

in which

R₁, R₂ and R₃, identical or different, independently represent:
hydrogen
a halogen
a C1 to C15 hydroxy or hydroxyalkyl group
a C1 to C15 linear or branched alkyl group a, b and c independently lying between 0 and 5.

R₄ is linked to the naphthalene unit at position 1, 2 or 3 via a —CH₂—O— bond and represents:
either a divalent group which is (co)polymerisable with a monomer; in which case d lies between 1 and 3
or a monovalent group which is (co)polymerisable with a monomer; in which case d is equal to 2 or 3 and

R₅ is linked to the naphthalene unit at position 1, 2 or 3 and represents:
hydrogen,
a halogen,
a C1 to C15 hydroxyalkyl group,
a C1 to C15 linear or branched alkyl group,
   and e is an integer lying between 0 and 2 and such that d+e=3.

What characterises the compounds of the invention and distinguishes them from compounds existing in the prior art is thus the presence of reactive substituents on the naphthalene group, which are linked to the aromatic ring by a —CH₂—O— bond, allowing these compounds to participate in chain or step (co)polymerisation reactions. This is made possible by the fact that the groups R₄ are such that at least one is a divalent group (co)polymerisable with a monomer or that at least two of these groups comprise a monovalent group (co)polymerisable with a monomer.

In the case of divalent polymerisable groups, the photochrome will be incorporated then anchored in the material by chain polymerisation (anionic, cationic, radical, ring opening, metathesis).

In the case of monovalent polymerisable groups, the photochromic material will be synthesised by polymerisation in steps between difunctional monomers or oligomers. Polyesters, polyethers, polyamides, polysiloxanes, etc. will then be formed by polycondensation or polyaddition. The other groups carried by the naphthalene unit (groups denoted by R₅) are inert groups and will therefore need to be non-reactive vis-à-vis the stepwise polymerisation reaction mechanism, and will be selected from among hydrogen, alkyls, halogens.

The groups R₁ and R₂ are advantageously selected independently from the group consisting of hydrogen, methyl, methoxy and fluorine, and R₃ and R₅ are advantageously hydrogen.

It will be readily understood that the monomers of the invention may be involved in chain polymerisation or step polymerisation reactions, depending on the nature of the polymerisable functions carried by the naphthalene ring.

More precisely, the monomer of the invention may be involved in a chain polymerisation reaction either when it is in the form of a copolymerisable divalent monomer or when it is in the form of a copolymerisable tetravalent monomer, which may then act as a crosslinking agent.

An example of a case in which the monomer of the invention behaves as a copolymerisable divalent monomer is that in which one of the three groups linked to positions 1, 2 or 3 of the naphthalene ring consists of the unit R₄—O—CH₂— in which R₄ comprises a vinyl function, an epoxide group, a (meth)acrylic group, a primary amino group, an anhydride and the two groups R₅ linked to positions 1, 2 or 3 of the naphthalene ring are selected from among hydrogen, a halogen, a C1 to C15 alkyl group, a C1 to C15 hydroxyalkyl group.

An example of a case in which the monomer of the invention behaves as a copolymerisable tetravalent monomer is that in which two of the groups linked to positions 1, 2 or 3 of the naphthalene ring consist of the unit R₄—O—CH₂— in which R₄ comprises a vinyl function, an epoxide group, a (meth)acrylic group, a primary amino group, an anhydride and the group R₅ is hydrogen, a halogen, a hydroxyl, a C1 to C15 alkyl group, C1 to C15 hydroxyalkyl and C1 to C15 alkoxy.

In the two cases above, the group or groups R₄ are advantageously selected from the group consisting of CH₂═CH—C(═O)—, CH₂═C(CH₃)—C(═O)—, —(CₙH₂ₙ)—OC(═O)CH═CH₂, —(CₙH₂ₙ)—OC(═O)C(CH₃)═CH₂, —(CH₂)_{n′}—CH═CH₂, —(CₙH₂ₙ)—O—(CH)_{n′}—CH═CH₂, —(CₙH₂ₙ)—O—CH═CH₂, and the group(s) R₅ is (are) H, (CₙH₂ₙ)—CH₃, n lying between 1 and 15 and n′ between 0 and 15.

Moreover, the monomers of the invention may be involved in step polymerisation reactions particularly when they are in the form of (co)polymerisable divalent monomers, i.e. when they carry 2 monovalent groups, or copolymerisable trivalent monomers capable of acting as a crosslinking agent.

An example of such (co)polymerisable divalent monomers is that of the monomers of the invention in which two of the groups linked to positions 1, 2 or 3 of the naphthalene ring consist of the unit R₄—O—CH₂— in which R₄ is hydrogen, a group carrying a carboxylic acid, C₁ to C15 hydroxyalkyl, an isocyanate, an epoxide, an amino, an anhydride, or a reactive silane group, in which case these groups may be identical or different, and the group R₅ is selected from among hydrogen or C₁ to C15 alkyl, a halogen.

An example of a copolymerisable trivalent monomer is that in which the three groups R₄ are selected independently from the group consisting of hydrogen, hydroxyalkyls, isocyanate, an anhydride, epoxides, aminos, groups carrying a carboxylic acid, an anhydride and groups carrying a reactive silane (Si—H or Si—C═C).

As an example of preferred monomers of the invention comprising at least two monovalent polymerisable groups, those will be mentioned in which at least two of the groups R₄ are selected from among hydrogen, the groups —(CₙH₂ₙ)—OH, —(CₙH₂ₙ)—NH₂, —(CₚH₂ₚ)—[CH—CH₂—O]cycle, —(CₙH₂ₙ)—COOH, —(CₙH₂ₙ)Si(CₘH₂ₘ)₂—H, —(CₙH₂ₙ)—Si—CH═CH₂, —(CₙH₂ₙ)Si(O—CₘH₂ₘ)₃, —C(═O)NH—R—N═C═O, with R═(CₙH₂ₙ), (CₙH₂ₙ₋₂), C5 to C20aryl, (CₙH₂ₙ₋₂)—CH₂—(CₙH₂ₙ₋₂) and aryl-CH₂-aryl, n and m lying between 1 and 15 and p lying between 0 and 15; the group R₅, if it is present, is H or C1 to C15 alkyl.

Among these compounds, those comprising three groups R₄ selected independently from the group as defined above make it possible to obtain copolymerisable trivalent monomers capable of acting as a crosslinking agent.

As regards the monomers of the invention comprising two monovalent polymerisable groups R₄, it should be noted that the polymerisable functions may be either of the same nature or of different natures, for example:
a hydroxyalkyl and an amine group, the group R₅ being hydrogen or an alkyl group,
a hydroxyalkyl group and a group carrying a carboxylic acid, the group R₅ being either hydrogen or an alkyl group,
a group carrying a carboxylic acid and an amine group, the group R₅ being either hydrogen or alkyl.

According to a second aspect, the invention also relates to a process for manufacturing the monomers to which the first aspect of the invention relates.

The person skilled in the art will readily understand that owing to the great variety of the substituents R₄, it is difficult to present a general synthesis scheme for all the products of the invention.

However, the common point of all the synthesis schemes envisaged is the fact that they present the advantage of comprising a cyclisation step, which will be referred below as a chromenisation, during which the intermediate product corresponding to formula II below is precipitated:

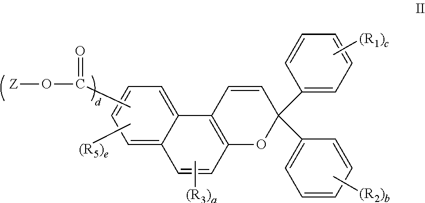

in which the group Z is either hydrogen or an alkyl group CₙH₂ₙ₊₁ with n=1 to 15 or a precursor, optionally protected, of the groups R₄, the groups R₁, R₂, R₃, R₄ and R₅ as well as a, b, c, d and e being as defined above.

This precipitated product will advantageously then undergo a reduction in step which will lead to the formation of the CH₂O bond, which connects the polymerisable group or groups to the naphthalene ring and which constitutes one of the essential characteristics of the products of the invention. Indeed, a key step is the selection of the correct solvent-nonsolvent pair making it possible to precipitate the product presenting the ester function. The person skilled in the art will of course understand that the nature of this pair may vary depending on the nature of the substituents, the one used in the synthesis schemes given below being particularly suitable for synthesising the described products.

Precipitating this intermediate presents the advantage of making it possible to obtain a high-purity product without having to resort to the purification steps that are necessary according to the prior art, which represents a particularly considerable industrial advantage.

The complete synthesis scheme will be given below in the case of the preferred monomers of the invention in which the group R₄ is a divalent polymerisable group of the (meth)acrylic type:

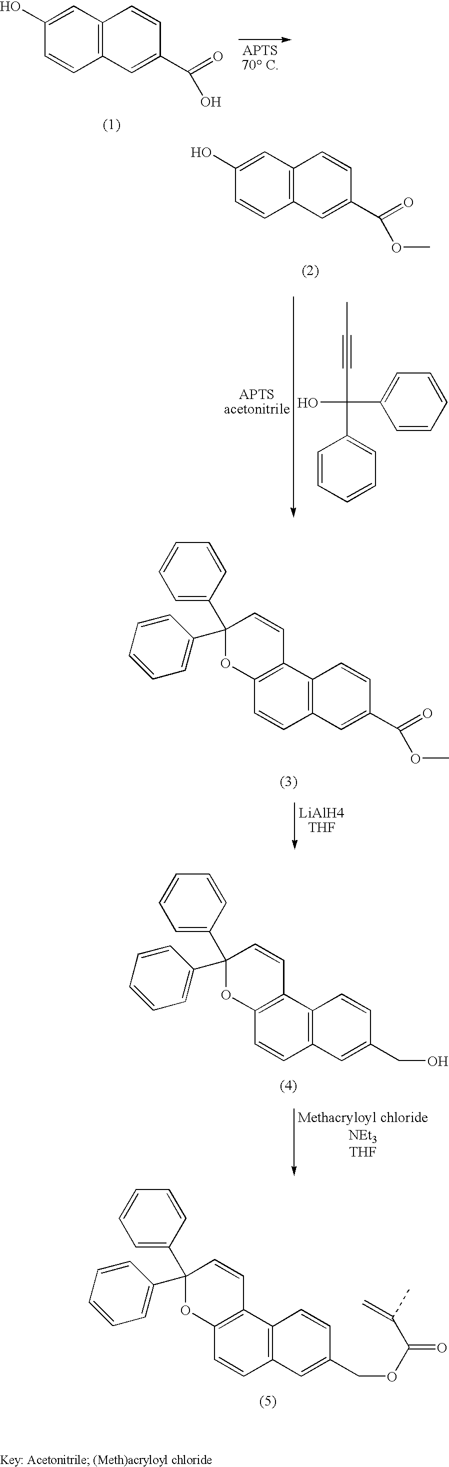

As shown on the synthesis scheme above, which forms the subject of a detailed description in Examples 1 and 2 which follow, the final product (product 5) is obtained by steps which are perfectly industrialisable owing to the good yield of each of these steps and the purity of the products obtained.

It will be noted that the product (3) can be isolated in a particularly simple and effective way by simple filtration, owing to the choice of the reaction medium in which the cyclisation (chromenisation) is carried out in order to synthesise it from the compound (2) of the previous step.

This step, during which the intermediate desired for the subsequent steps is precipitated, proves to be a key step of the method.

It is in fact from this product (3) that a good number of monomers of the invention can be manufactured.

Specifically, in the case of the scheme above, the compound (3) is subjected to a reduction step in order to lead to the product (4) which then undergoes grafting.

The person skilled in the art will readily understand in view of this scheme, which comprises perfectly conventional chemical operations, that other “deprotection” steps may be envisaged, for example hydrolysis of the ester function in order to obtain a carboxylic acid or controlled reduction of the ester to an aldehyde.

Other grafting steps may also be envisaged, for example reaction of the hydroxylated product (4) with a diisocyanate in order to obtain a photochrome with a reactive isocyanate function, or functionalisation of the product (4) by a transetherification reaction with an enolic ether in the presence of mercury acetate in order to lead to a vinyl function.

The person skilled in the art will moreover readily understand that, in a scheme similar to that represented above, an epoxy group may be obtained by reacting the compound (4) with epichlorhydrin.

As regards the monomers of the invention comprising two monovalent groups, they may be obtained by following the scheme above or a scheme derived from this scheme which is readily envisageable by the person skilled in the art.

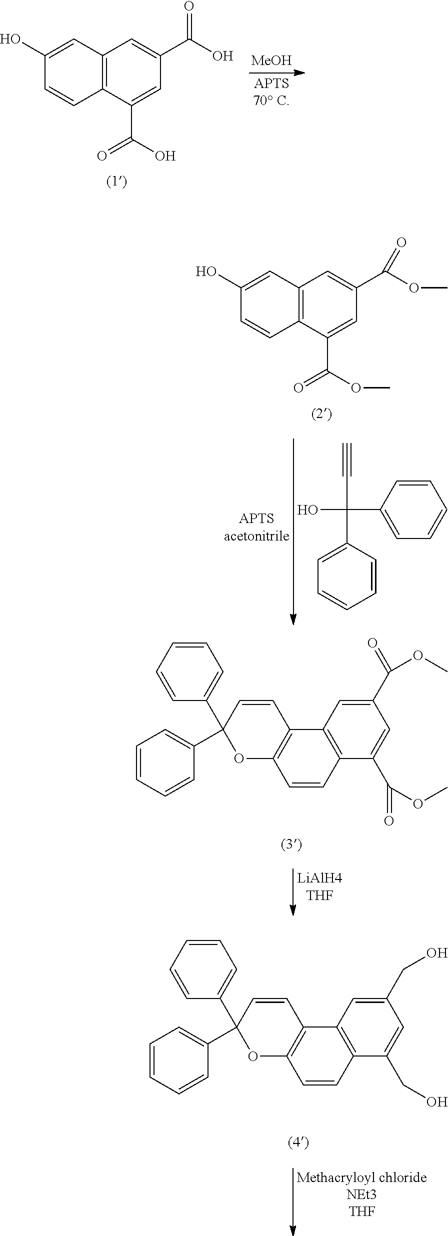

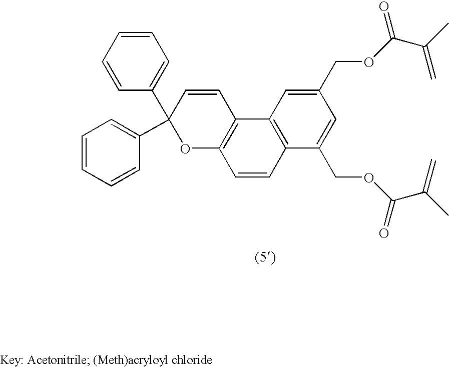

As in the previous case, other types of functions may be envisaged; for example, the hydroxyl group of the compound (4′) may be transformed into an amine or the ester (3′) may be hydrolysed to a carboxylic acid.

According to its third aspect, the invention relates to polymers obtained by copolymerisation of a compound of the invention with a monomer.

Virtually all existing monomers may be copolymerised with the monomers of the invention. Copolymerisation of the monomers of the invention with other known photochromic monomers could also be envisaged. For example, the one described by Transition Optical in WO 03/056390 carrying a methacrylate function could be copolymerised with the monomer of the invention and methyl methacrylate in order to manufacture photochromic organic glasses.

As nonlimiting examples of comonomers which may be envisaged, those which carry one or more hydroxyl, amino, (meth)acrylate, vinyl, epoxy, isocyanate, anhydride, acid, silane functions will be mentioned, or a mixture of different monomers.

The photochromic polymer which is obtained presents a decolouration rate and a quantum efficiency which are similar to that of the photochrome forming the subject of the first aspect of the invention.

Thus, it is possible to synthesise hydrophobic polymers for implants, for varnishes, hydrophilic or amphiphilic polymers for creams etc.

For example, the photochrome carrying a single hydroxyl function (R₄=—H), and R₁ to R₃=H may react with a mixture of diol and diisocyanate in order to obtain a photochromic polyurethane.

For example, the photochrome carrying a single acrylic function (R₄=—C(═O)CH═CH₂), and R₁ to R₃=H may be copolymerised with butyl acrylate and styrene in order to manufacture a hydrophobic element which protects against UV when it is exposed to sunlight.

For example, the photochrome carrying a single methacrylic function R₄=—C(═O)CH═CH(CH₃), and R₁ to R₃=H may be copolymerised with 2-hydroxyethyl methacrylate or acrylamide in order to manufacture a water-soluble polymer involved in the composition of aqueous gels which become coloured by UV.

Attachment of the monomers of the invention onto existing oligomers or polymers could also be envisaged. For example, the monomer of the compound (4) type carrying both a group R₄=—H and R₄=acrylate could be grafted by esterification onto a polyacid and lead to crosslinking of the latter by radical post-polymerisation of the acryloxy group. Numerous oligomers or polymers could thus be chemically modified by the photochromic polymers of the invention, and thus become photosensitive.

The possibilities are very wide; the following examples are not limiting, rather they are only an illustration of the invention.

### EXAMPLES

### Example 1

### Synthesis of 8-hydroxymethyl-3,3 diphenyl-3H naphtho[2,1]-pyrane (compound (4))

This example is given with reference to the 1^{st} synthesis scheme.

### Step 1: Esterification of Compound (1)

Compound (1) (7 g) is dissolved in 80 ml of methanol in a three-necked round-bottomed flask (250 ml) with a condenser on top. The esterification reaction is catalysed by adding para-toluenesulfonic acid (APTS, 0.48 g) introduced under nitrogen. The reaction is conducted at a temperature of 70° C. and left to stir for at least 12 hours.

At the end of this time, the methanol is evaporated and the residual organic phase is dissolved in ethyl acetate with a view to liquid/liquid extractions (ethyl acetate/water saturated with potassium carbonate) in order to purify the intended ester (compound (2)).

The organic phase resulting from the various washes is subsequently dried on MgSO₄ then filtered. Evaporation of the ethyl acetate allows the desired ester to be isolated quantitatively (6 to 7 g).

### Step 2: Chromenisation of Compound (2)

In a three-necked round-bottomed flask (100 ml) with a condenser on top, 1.56 g of compound (2) is introduced with 50 ml of acetonitrile. The medium becomes limpid as soon as the temperature of 50° C. is reached, at which moment 1.6 g (1 eq/(2)) of propargylic alcohol and 0.122 g of APTS (0.08 eq/(2)) are added under nitrogen. The reaction medium is cooled to room temperature and stirred for two days at this temperature.

Compound (3) is simply isolated by filtration since it is insoluble in the reaction medium. It is purified by washes in acetonitrile at 40° C. followed by filtrations. The product is obtained with a yield of 55% without additional purification.

Step 3: Reduction of the ester (3) in order to obtain (4): 8-hydroxymethyl-3,3 diphenyl-3H naphtho[2,1]-pyrane

In a three-necked round-bottomed flask (100 ml) fitted with a bubbler, 0.2 g of LiAlH₄ (1.41 eq/(3)) diluted in 15 ml of anhydrous THF is introduced. The 1.5 g of (3) dissolved in 30 ml of anhydrous THF are then added dropwise under nitrogen. No violent release of gas is observed during the addition. Everything is stirred for at least 12 hours at room temperature.

Before isolating product (4), the excess LiAlH₄ must be neutralised. In order to do this, 1.25 ml of water then 1.25 ml of a 10% strength sulphuric acid solution (H₂SO₄) are added slowly. Adding ether to the medium makes two phases appear. The extracted organic phase is washed (water saturated with NaCl) then dried with MgSO₄, filtered and the solvent is evaporated.

The white product, obtained with a quantitative yield, corresponds to the intended compound (4).

### Example 2

### Functionalisation with Acryloyl Chloride: Compound (5)

In a three-necked round-bottomed flask (100 ml) with a condenser on top, 1 g of compound 4, 0.5 ml of Et₃N (1.3 eq/(4)) dissolved in 30 ml of anhydrous THF are introduced. The chloride is added dropwise under nitrogen at room temperature, without observing a significant rise in temperature. The reaction is left at room temperature while stirring for at least 12 hours.

At the end of this period, the solvent is evaporated. The residue is diluted in dichloromethane then extracted by successive washes with water saturated with potassium carbonate. The extracted organic phase is dried with MgSO₄, filtered on silica and the solvent is evaporated.

The molecule (5) is isolated with a yield of 60% by weight.

The following table collates the results obtained in terms of quantum efficiency, decolouration rate and fatigue strength of the product obtained, in comparison with various products of the literature.

| | Colour in | Quantum | Decoloura- | Fatigue |
|---|---|---|---|---|
| Reference | excited form | efficiency | tion rate | strength |
| diphenyl- | yellow- | 0.3-0.4 | <200 | good |
| naphthopyrane | orange | | | |
| (invention) | | | | |
| naphthopyranes | yellow- | 0.2-0.7 | 40-270 | good |
| (U.S. Pat. No. | green-blue | (UV/90 s) | | |
| 6,113,814) | | | | |
| dinaphtho-pyranes | blue-violet | 0.2 | 150-600 | good |
| (U.S. Pat. No. | | (UV/5 s) | | |
| 5,464,567) | | | | |
| indeno-naphtho- | yellow- | 0.3-0.7 | 50-100 | average |
| pyrane | brown | (UV/15 min) | | |
| (WO03056390, | | | | |
| W09614596, | | | | |
| polymer matrix) | | | | |

Quantum efficiency: efficiency at absorbing UV rays in order to make the ring open at 22° C., over 5 seconds of irradiation.
Decolouration rate: rate at which the colour disappears when the UV stops; here, this rate is represented by the time at the end of which the quantum efficiency falls to 50% (in seconds); in certain cases, it was measured in a polymer matrix which generally changed a t_{1/2} from 50 to 100 s.
Fatigue strength: quality of the photochrome to perform numerous colouration/decolouration cycles; a good fatigue strength corresponds to a 5% loss of efficiency after about 50 cycles.

### Example 3

### Functionalisation with Methacryloyl Chloride

The synthesis is identical to that of Example 2, except that the acryloyl chloride is replaced by methacryloyl chloride.

### Example 4

### Synthesis of a Polyurethane from Product (4) of Example 1 and a Diisocyanate

In a one litre reactor with a condenser on top, 400 ml of dried methyl ethyl ketone (MEK) and 50 g of dried dihydroxyl polyethylene oxide (PEO) (Mn=200 g/mol, i.e. 0.05 mol of OH functions) and 26.2 g of 4,4′-methylenebiscyclohexyl diisocyanate (M=262 g/mol, i.e. 0.1 mol of NCO functions) are dissolved under a flow of nitrogen. The reactor is heated by a double jacket to reflux of the solvent (70° C.) then a catalytic quantity of tin dibutyldilaurate is added.

When the consumption of isocyanate no longer changes under monitoring by infrared spectroscopy, the photochromic molecule 1 (19.4 g, i.e. 0.05 mol) is added in order to terminate the chains.

The polymer obtained is recovered by precipitation in a mixture of oils with an excellent yield (>90%). When dissolved in water, it reversibly develops the orange colouration in a few seconds under UV.

### Example 5

### Synthesis of a Hydrophobic Acrylic Polymer from Compound (5), Styrene and Butyl Acrylate

In a 250 ml round-bottomed flask with a condenser on top, 100 ml of distilled tetrahydrofuran (THF) is introduced and 70 g of distilled styrene, 30 g of distilled butyl acrylate, 0.2 g of the molecule (5) obtained in Example 2, then 0.2 g of 2,2′-azobisisobutyronitrile are dissolved, nitrogen is bubbled through in order to degas the reaction mixture, then the round-bottomed flask is heated to 60° C. for 4 h.

The polymer is recovered by precipitation in ethanol with a yield of 80% by weight.

When put into the form of a coating on a glass plate, the polymer develops an immediate orange-yellow colouration under UV and decolours in 20 seconds when shaded from the light.

### Example 6

### Synthesis of a Hydrophilic Methacrylic Polymer from the Product of Example 3 and Hydroxyethyl Methacrylate

In a 250 ml round-bottomed flask with a condenser on top, 100 g of distilled hydroxyethyl methacrylate, 0.5 g of ethylene glycol dimethacrylate, 0.2 g of the molecule (5) obtained in Example 3, then 0.2 g of 2,2′-azobisisobutyronitrile are introduced. Nitrogen is bubbled through in order to degas the viscous reaction mixture then the solution obtained is poured into a mould, for example a small tube. This mould is heated to 50° C. for 12 hrs then 60° C. for 4 hrs and finally 80° C. for 4 hrs.

The rod-shaped polymer is baked in an oven for 12 hrs at 80° C. When it is soaked with water, it swells and reversibly develops the colour yellow when it is exposed to sunlight.

### Example 7

### Photopolymerisation of the Product of Example 2 and Benzyl Acrylate

In a mixture of benzyl acrylate (99%), hexanediol diacrylate (HDDA, 1%) containing 0.2% of 2,2-dimethoxy-1,2-diphenylethan-1-one or 1-hydroxy cyclohexyl phenyl ketone, 0.5% by weight of the monomer of Example 2 is added. After degassing by bubbling nitrogen through for 15 minutes, the mixture is placed in a glass mould and irradiated with ultraviolet lamps for 300 to 1,200 seconds, depending on the lamp.

The film obtained develops an immediate orange-yellow colouration under renewed UV stimulation and decolours rapidly when shaded from the light.

1. Compound of the polymerisable substituted 3,3-diaryl-3H-naphtho[2,1-b]pyrane type, characterised in that it corresponds to formula (I): 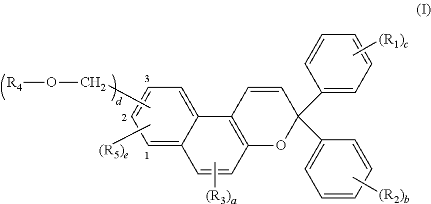
in which
R₁, R₂ and R₃, are identical or different, and independently represent:
hydrogen
a halogen
a C1 to C15 hydroxy or hydroxyalkyl group
a C1 to C15 linear or branched alkyl group a, b and c independently lying between 0 and 5
R₄ is linked to the naphthalene unit at position 1, 2 or 3 via a —CH₂—O— bond and represents:
either a divalent group which is (co)polymerisable with a monomer; in which case d lies between 1 and 3
or a monovalent group which is (co)polymerisable with a monomer; in which case d is equal to 2 or 3 and
R₅ is linked to the naphthalene unit at position 1, 2 or 3 and represents:
hydrogen,
a halogen,
a C1 to C15 hydroxyalkyl group,
a C1 to C15 linear or branched alkyl group,
and e is an integer lying between 0 and 2 and such that d+e=3.

2. Compound according to claim 1, wherein one of the three groups linked to positions 1, 2 or 3 of the naphthalene ring consists of the unit R₄—O—CH₂— in which R₄ comprises a vinyl function, an epoxide group, a (meth)acrylic group, a primary amino group, an anhydride and the two groups R₅ linked to positions 1, 2 or 3 of the naphthalene ring are selected from among hydrogen, a halogen, a C1 to C15 alkyl group, a C1 to C15 hydroxyalkyl group, C1 to C15 alkoxy.

3. Compound according to 2, wherein two of the groups linked to positions 1, 2 or 3 of the naphthalene ring consist of the unit R₄—O—CH₂— in which R₄ comprises a vinyl function, an epoxide group, a (meth)acrylic group, a primary amino group, an anhydride and the group R₅ is hydrogen, a halogen, a C1 to C15 alkyl group, C1 to C15 hydroxyalkyl.

4. Compound according to claim 3, wherein one of the two groups linked to positions 1, 2 or 3 of the naphthalene ring is (are) the unit R₄—O—CH₂ with R₄ selected from the group consisting of CH₂═CH—C(═O)—, CH₂═C(CH₃)—C(═O)—, —(CₙH₂ₙ)—OC(═O)CH═CH₂, —(CₙH₂ₙ)—OC(═O)C(CH₃)═CH₂, —(CH₂)_{n′}—CH═CH₂, —(CₙH₂ₙ)—O—(CH)_{n′}—CH═CH₂, —(CₙH₂ₙ)—O—CH═CH₂, and the group(s) R₅ is (are) H, (CₙH₂ₙ)—CH₃, (CₙH₂ₙ)—OH, n lying between 1 and 15 and n′ between 0 and 15.

5. Compound according to claim 2, wherein two of the groups linked to positions 1, 2 or 3 of the naphthalene ring consist of the unit R₄—O—CH₂— in which R₄ is hydrogen, a group carrying a carboxylic acid, C₁ to C15 hydroxyalkyl, an isocyanate, an epoxide, an amino, an anhydride, or a reactive silane group, in which case these groups may be identical or different, and the group R₅ is selected from among hydrogen, C1 to C15 alkyl, a halogen.

6. Compound according to claim 2, wherein the three positions 1, 2 and 3 of the naphthalene unit are substituted by the unit R₄—O—CH₂— with R₄ selected independently from the group consisting of hydrogen, hydroxyalkyls, isocyanate, an anhydride, epoxides, aminos, groups carrying a carboxylic acid and groups carrying a reactive silane.

7. Compound according to claim 6, wherein at least two of the positions 1, 2 or 3 of the naphthalene unit carries a R₄—O—CH₂— group with R₄ selected from the group consisting of hydrogen, —(CₙH₂ₙ)—OH, —(CₙH₂ₙ)—NH₂, —(CₚH₂ₚ)—[CH—CH₂—O] ring, —(CₙH₂ₙ)—COOH, —(CₙH₂ₙ)—Si(CₘH₂ₘ)₂—H and —(CₙH₂ₙ)—Si—CH═CH₂, —(CₙH₂ₙ)—Si—(O—CₘH₂ₘ)₃, —C(═O)NH—R—N═C═O, with R═(CₙH₂ₙ) or (CₙH₂ₙ₋₂) or —(CₙH₂ₙ₋₂)—CH₂—(CₙH₂ₙ₋₂) or C5 to C20 aryl and aryl-CH₂-aryl, n and m lying between 1 and 15 and p lying between 0 and 15; the group R₅, if there is one, is H or C1 to C15 alkyl.

8. Compound according to claim 7, wherein the three substituents carried at positions 1, 2 and 3 of the naphthalene unit are of the form R₄—O—CH₂— with R₄ selected independently from the group consisting of hydrogen, —(CₙH₂ₙ)—OH, —(CₙH₂ₙ)—NH₂, —(CₙH₂ₙ)—COOH, —(CₚH₂ₚ)—[CH—CH2-O] ring, (CₙH₂ₙ)—Si(CₘH₂ₘ)—H, (CₙH₂ₙ)—Si(O—CₘH₂ₘ)₃, —C(═O)NH—R—N═C═O, with R═(CₙH₂ₙ) or (CₙH₂ₙ₋₂) or —(CₙH₂ₙ₋₂)—CH₂—(CₙH₂ₙ₋₂) or C5 to C20 aryl and aryl-CH₂-aryl, with n and m lying between 1 and 15 and p lying between 0 and 15;

9. Compound according to claim 1, which corresponds to the formula: 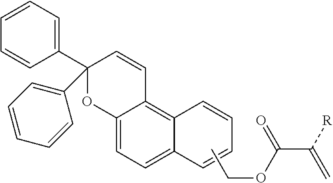
with R═H or CH₃

10. Process for synthesising a compound according to claim 1, which comprises a step of cyclisation, so-called chromenisation step, during which the intermediate product corresponding to formula II below is precipitated in the reaction medium: 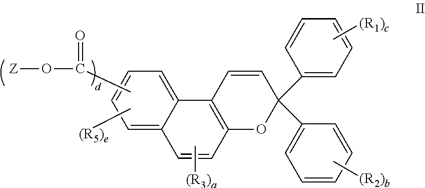
in which the group Z is either hydrogen or an alkyl group CₙH₂ₙ₊₁ with n=1 to 15 or a precursor, optionally protected, of the groups R₄, the groups R₁, R₂, R₃, R₄ and R₅ as well as a, b, c, d and e being as defined in the preceding claims.

11. A method of preparation of a polymer, a copolymer or an oligomer with a photochromic or photosensitive property comprising use of a compound according to claim 1.

12. The method according to claim 11, wherein the said compound is used as a monomer or comonomer in a polymerisation or copolymerisation reaction intended to produce a polymer or a copolymer with a photochromic or photosensitive property.

13. The method according to claim 12, wherein the said compound is used to chemically modify an oligomer or a polymer and render it photosensitive.

14. Compound according to claim 1, wherein the groups R₁ and R₂ are selected independently from the group consisting of hydrogen, methyl, methoxy and fluorine, and in that R₅ and R₃ are hydrogen.
